(19) United States
(12) Patent Application Publication
(10) Pub. No.: US 2007/0219141 A1
(43) Pub. Date: Sep. 20, 2007

(54) Plant Materials Extraction Method

(76) Inventors: David Jones, Burnaby (CA); Kenneth John Falk, Saskatoon (CA)

Correspondence Address: KLARQUIST SPARKMAN, LLP, 121 SW SALMON STREET, SUITE 1600, PORTLAND, OR, 97204, US

(21) Appl. No.: 10/582,317

(22) PCT Filed: Dec. 7, 2004
(86) PCT No.: PCT/CA04/02087

§ 371 Date: Apr. 26, 2007

Related U.S. Application Data
(60) Provisional application No. 60/527,302, filed on Dec. 8, 2003.

(51) Int. Cl. A61K 31/122; A61K 31/00; C07C 49/307; C07G 1/00; C09F 1/02; C11B 9/02; C11D 3/20; C11D 3/382

(52) U.S. Cl. 514/22; 510/464; 510/505; 512/5; 514/675; 530/202; 530/507; 568/375

(57) ABSTRACT

A process for preparing plant extracts is provided. Plant materials, including tropolones, lignins and polar molecules, are mixed with a liquid polar solvent to form an extraction mixture, which is maintained under extraction conditions effective to extract lignins, polar molecules and at least 50% of the tropolones into the polar solvent to form a pregnant polar solvent liquid phase. The pregnant polar solvent phase is separated from the solid plant materials, and mixed with a substantially immiscible nonpolar solvent under conditions effective to partition the tropolones and lignins substantially into the nonpolar solvent and to partition the polar molecules substantially into the polar solvent to form a partitioned nonpolar solvent phase comprising lignins and tropolones, and a partitioned polar solvent phase comprising the polar molecules, and separating the polar solvent phase from the nonpolar solvent phase to obtain a polar plant extract and a nonpolar plant extract.

### FIELD OF INVENTION

Aspects of the invention relate to methods of extracting organic compounds from solid plant materials, and more specifically, to methods of extracting organic compounds from solid plant materials using liquid solvents, and the extracts obtainable by such methods.

### BACKGROUND OF THE INVENTION

Thuja plicata Don., commonly known as the Western red cedar, is a North American tree of the Cupressaceae family (Order Cupressales) native to the Pacific Northwest. Extensively harvested for wood products, the tree has also been shown to contain compounds of biological interest (see, for example, “The Chemistry and Utilization of Western Red Cedar” by Barton, G. M., et al. Publication 1023 of the Government of Canada, Department of Fisheries and Forestry, 1971; and U.S. Pat. No. 4,645,536). Western red cedar leaf, wood and bark oils have been found to contain a number of biologically active substances, some of which have been characterized. There are for example several components in Western Red Cedar heartwood, such as alpha-thujaplicin, gamma-thujaplicin, beta-thujaplicin (also known as “hinokitiol”, see for example U.S. Pat. No. 5,658,584, and 2,4,6-Cycloheptatrien-1-one,2-hydroxy-4-(1-methylethyl) [499-44-5]), beta-dolabrin, beta-thujaplicinol, thujic acid, tropolone (2-Hydroxy-2,4,6-cycloheptatrien-1-one [533-75-5]) and methyl thujate. Many of these compounds are members of a class of compounds known as tropolones, which are 2-hydroxycyclohepta-2,4,6-trienones and derivatives formed by substitution.

Some of the compounds identified in Western red cedar extracts have been found in the heartwood of other decay-resisting species of trees, particularly in the Cupressaceae species. In Western red cedar, tropolones may comprise a small percentage of the tree components. Other substances in cedar heart wood may include non-volatile substances such as plicatic acid, plicatin, thujaplicatin, lignins and cellulose. Some of these compounds, particularly plicatic acid, have been implicated in the development of asthma (Chan-Yeung, M. (1994) Am J Ind Med. 25(1):13-8).

Tropolone components have been studied as antibacterial agents, see for example by Anderson, A B et al. Acta Chem. Scand. (1948) 2:644; Erdtman, H. et al. Nature (1948),161:719; Gripenberg, J. Acta Chem. Scand. (1948) 2, 639; Arima, Y; Nakai Y; Hayakawa R. et al. (2003), J Antimicrob Chem 51(1):113-22; Inamori, Y. Shinohara, S., Tusjibo, H. et al. (1999), Biological & Pharmaceutical Bulletin, 22(9): 990-3.

“Waste wood” is a term that may be used to characterize cellulosic material comprising wood shavings, sawdust, bark, splinters, etc., which are frequent byproducts of the lumber industry. In many circumstances, waste wood is an underutilized commodity that may present disposal issues. In addition, the potentially useful chemical components of such species as Western red cedar are relatively inaccessible in waste wood, as they are contained in small quantities entrained in wood fibres with other compounds.

### SUMMARY

In accordance with one aspect of the invention, there is provided a process for preparing extracts from solid plant materials. The plant materials may, for example, comprise tropolones, lignins and/or and polar molecules, and the process may include mixing the plant materials with a liquid polar solvent to form an extraction mixture. The extraction mixture may, for example, be maintained under extraction conditions effective to extract a proportion of the lignins, such as 50%, the polar molecules and a proportion of the tropolones, such as 50%, in the plant materials into the polar solvent to form a pregnant polar solvent liquid phase and a solid phase of extracted plant materials in the extraction mixture. The pregnant polar solvent liquid phase may then be separated from the solid plant materials, and may be mixed with a substantially immiscible nonpolar solvent under partition conditions effective to partition the tropolones and lignins substantially into the nonpolar solvent, and to partition the polar molecules substantially into the polar solvent, forming a partitioned nonpolar solvent phase including lignins and tropolones, and a partitioned polar solvent phase including the polar molecules. The partitioned polar solvent phase may then be separated from the partitioned nonpolar solvent phase to obtain a polar plant extract and a nonpolar plant extract.

In accordance with another aspect of the invention, there is provided a process for preparing extracts from solid plant materials, the plant materials may for example include tropolones, lignins and/or plicatic acid. The process may include mixing the plant materials with a liquid polar solvent to form an extraction mixture. The extraction mixtures may be maintained under extraction conditions effective to extract a proportion of the plicatic acid, a proportion of the lignins and a proportion of the tropolones, such as 50%, in the plant materials into the polar solvent to form a pregnant polar solvent liquid phase and a solid phase of extracted plant materials. The pregnant polar solvent liquid phase may be separated from the solid plant materials. The pregnant polar solvent liquid phase may be mixed with a nonpolar solvent which may be substantially immiscible, under partition conditions to partition the tropolones and the lignins substantially into the nonpolar solvent, and to partition the plicatic acid substantially into the polar solvent, to form a partitioned nonpolar solvent phase including lignins and tropolones and a partitioned polar solvent phase including plicatic acid. The partitioned polar solvent phase may then be separated from the partitioned nonpolar solvent phase to obtain a polar plant extract and a nonpolar plant extract.

The processes described above may further include concentrating the partitioned polar solvent phase and the partitioned nonpolar solvent phase by removing the polar and the nonpolar solvents respectively to form a concentrated polar phase and a concentrated nonpolar phase. The polar solvent and the nonpolar solvent may, for example, be removed by distillation.

The concentrated nonpolar plant extract may be treated with an additional wash of a nonpolar solvent effective to partition lignins and tropolones into a lignin extract and a tropolone extract. The nonpolar solvent may be diethyl ether.

The plant materials may be wood, which may be from a plant species selected from the plant order Cupressales, and may include Chamaecyparis formosensis, Chamaecyparis Iawsoniana, Chamaecyparis obtusa, Chamaecyparis taiwanensis, Chamaecyparis thyoides, Cupressus abramsiana, Cupressus arizonica, Cupressus bakeri, Cupressus goweniana, Cupressus macnabiana, Cupressus macrocarpa, Cupressus pygmaea, Cupressus sargentii, Cupressus sempervirens, Cupressus torulosa, Juniperus cedrus, Juniperus communis, Juniperus chinensis, Juniperus deppeana, Juniperus monosperma, Juniperus osteosperma, Juniperus phoenicea, Juniperus thurifera, Juniperus utahensis, Calocedrus decurrens, Calocedrus formosana, Platycladus orientalis, Thuja occidentalis, Thuja plicata Don., Thuja standishii, Thujopsis dolabrata, Tetraclinis articulata, and/or Austrocedrus chilensis.

The polar solvent may be a liquid solvent having a polarity index of at least 4. The polar solvent may be 2-methyl-i-propanol, methyl isoamyl ketone, n-butyl acetate, methyl isobutyl ketone, tetrahydrofuran, 2,6-lutidine, ethyl acetate, isopropanol, chloroform, cyclohexanone, methyl ethyl ketone, methyl n-propyl ketone, 2-picoline, dioxane, ethanol, nitroethane, pyridine, acetone, methoxyethanol, acetic acid, acetonitrile, methanol, nitromethane, m-cresol; and/or water.

The nonpolar solvent may be a liquid solvent having a polarity index less than 4. The nonpolar solvent may be squalane, isooctane, n-decane, 1,1,2-trichlorotrifluoroethane, cyclohexane, n-hexane, pentane, cyclopentane, heptane, petroleum ether, carbon disulfide, n-butyl chloride, carbon tetrachloride, dibutyl ether, triethylamine, diisopropyl ether, toluene, o-xylene, p-xylene, methyl t-butyl ether, bromobenzene, chiorobenzene, iodobenzene, o-dichlorobenzene, diethyl ether, benzene, dichloromethane, ethyl bromide, fluorobenzene, ethylene dichloride, isopentanol, ethylene chloride, 2-propanol, n-butanol, n-propanol, and/or tert-butanol.

The extraction conditions in the processes may be maintained for an extraction period of from about one minute to three days, from about one to 24 hours; about 24 hours, from about four to 12 hours; about four hours, about six hours, or about 12 hours. The extraction conditions may be cycled or repeated.

In accordance with an aspect of the invention, there is provided a polar plant extract. Preservatives, antioxidants, plastics, cleansing agents, and disinfecting agents having the polar plant extract as a component are provided.

In accordance with an aspect of the invention, there is provided a nonpolar extract. The nonpolar plant extract may be used as components of preservative compositions, antioxidant compositions, fragrances, cleansing agents, and disinfecting agents. The nonpolar extracts may be used in the manufacture of medicaments for treating infection, fragrances, antibacterial agents, anticancer agents, antifungal agents, insecticidal agents, cleansing agents, and disinfecting agents.

In accordance with an aspect of the invention, there is provided extracted plant materials, which may be used for the construction of hypoallergenic wood products.

In accordance with an aspect of the invention, there is provided a lignin extract. The lignin extract may be used in the manufacture of antioxidizing agents, or act as an antioxidant ingredient.

In accordance with an aspect of the invention, there is provided a tropolone extract. The tropolone extract may be used in the manufacture of medicaments for treating infection, disinfecting agents, fragrances, antibacterial agents, anticancer agents, antifungal agents, and insecticidal agents. The tropolone extract may be used in the treatment of disorders including infection such as by antibiotic resistant bacteria or fungi such as C. albicans.

Methods of treating infection using the extracts of the invention are also provided.

### BRIEF DESCRIPTION OF THE DRAWINGS

In drawings which illustrate embodiments of the invention,

FIG. 1 is a schematic of an extraction scheme according to one embodiment of the invention;

FIG. 2A is an HPLC trace for a column Fraction 22 of a nonpolar extract according to one embodiment of the invention using three 24 hour passes of methanol, and five passes of DCM.

FIG. 2B is an HPLC trace for Fraction 18 as described for FIG. 2A;

FIG. 2C is an HPLC trace for Fraction 14 as described for FIG. 2A;

FIG. 2D is an HPLC trace for Fraction 9 as described for FIG. 2A; and

FIG. 2E is an HPLC trace for Fraction 5 as described for FIG. 2A.

### DETAILED DESCRIPTION

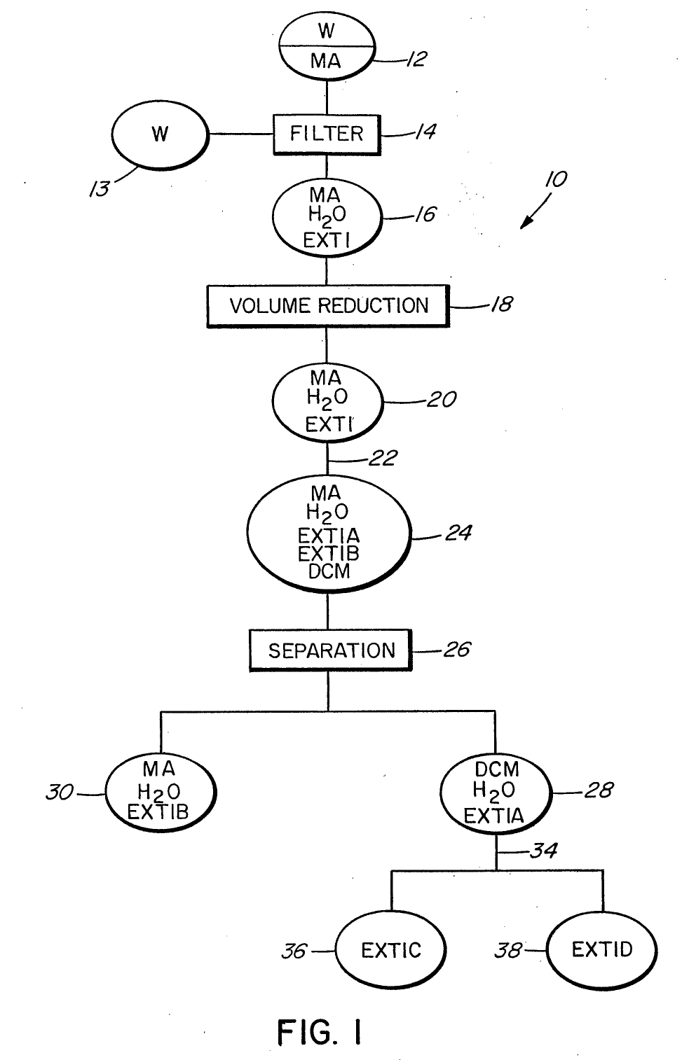

Referring to FIG. 1, the process according to a first embodiment of the invention is shown generally at 10.

In a process for preparing extracts from solid plant materials, plant materials which may comprise tropolones, lignins and/or polar molecules are combined with a liquid polar solvent to form an extraction mixture 12, which may then be maintained under extraction conditions effective to extract the a proportion of the lignins, the polar molecules and the tropolones, such as 50% of these components, from the plant materials into the polar solvent to form a pregnant polar solvent liquid phase and a solid phase of extracted plant materials in the extraction mixture.

A polar solvent may be a compound that is composed of polar molecules. Polar solvents can dissolve ionic compounds or covalent compounds that ionize. A polar compound may be defined as any compound with a polarity index of 4 or higher. Polarity index is a relative measure of the degree of interaction of the solvent with various polar test solutes. A useful reference is L R Snyder's 1978 “Classification of the Solvent Properties of Common Liquids” in The Journal of Chromatography Science, 16, 223, (1978) incorporated herein by reference.

The pregnant polar solvent liquid phase may be separated 14 from the solid plant materials 13, by filtration or layering as preferred and further described below. The pregnant polar solvent liquid phase 16 may be concentrated 18 at this point by distillation or other means. The pregnant polar liquid solvent 16 may then be mixed with a nonpolar solvent, which may be substantially immiscible, to partition the tropolones and lignins substantially into the nonpolar solvent, and to partition the polar molecules substantially into the polar solvent, forming a partitioned nonpolar solvent phase including a proportion of the lignins and tropolones, and a partitioned polar solvent phase including a proportion of the polar molecules.

In some embodiments, partition conditions include room temperature and normal atmospheric pressure, but may also include reduced atmospheric pressure and temperatures ranging from 0 to about 200° C.

Nonpolar solvents may be compounds that will only dissolve nonpolar covalent compounds. A nonpolar compound may be classified as any compound with a polarity index of 4 or lower.

The partitioned polar solvent phase may be separated from the partitioned nonpolar solvent phase to obtain a polar plant Extract 1B 30 and a nonpolar plant Extract 1A 28.

The polar solvent and nonpolar solvents may be removed after extraction to form a concentrated polar phase and a concentrated nonpolar phase. The polar solvent and the nonpolar solvent may, for example, be removed by distillation, chromatography, or removed as a layer after settling or centrifugation.

Solid phase separation techniques may be used to remove the solvents, and to separate and purify specific compounds found in the extracts of the invention. These may include various types of chromatography, including silica gel columns, TLC, preparatory TLC, and HPLC.

The nonpolar plant Extract 1A 28, concentrated or not, may then be treated with an additional wash of a nonpolar solvent 34 effective to partition lignins and tropolones into a Lignin Extract 1D 38 and a Tropolone Extract 1C 36. The nonpolar solvent may be diethyl ether but is not limited thereto.

The plant materials used are generally wood from a plant species selected from the plant order Cupressales, and may include one or more of Chamaecyparis formosensis, Chamaecyparis lawsoniana, Chamaecyparis obtusa, Chamaecyparis taiwanensis, Chamaecyparis thyoides, Cupressus abramsiana, Cupressus arizonica, Cupressus bakeri, Cupressus goweniana, Cupressus macnabiana, Cupressus macrocarpa, Cupressus pygmaea, Cupressus sargentii, Cupressus sempervirens, Cupressus torulosa, Juniperus cedrus, Juniperus communis, Juniperus chinensis, Juniperus deppeana, Juniperus monosperma, Juniperus osteosperma, Juniperus phoenicea, Juniperus thurifera, Juniperus utahensis, Calocedrus decurrens, Calocedrus formosana, Platycladus orientalis, Thuja occidentalis, Thuja plicata, Thuja standishii, Thujopsis dolabrata, Tetraclinis articulata, and Austrocedrus chilensis.

The starting plant materials may include not just tropolones and lignins, but also plicatic acid, a polar molecule. Plicatic acid may then be sequestered in Extract 1B 30. Thuja plicata Don. is a useful plant material for the production of many desired compounds as well as plicatic acid by processes of the invention. Plicatic acid finds use as either an additive or a sole ingredient for new plastic materials for food packaging and industrial use.

Plant materials may be macerated, chipped, chopped, ground, cut into smaller pieces, ground up, crushed, pulverized, or splintered, etc. Plant materials, including wood, may be a natural by product of normal lumber or crop processing, or may be specifically harvested and processed for the purpose of extraction.

The polar solvent may be a liquid solvent having a polarity index of at least 4. The polar solvent may be one or more of 2-methyl-1-propanol, methyl isoamyl ketone, n-butyl acetate, methyl isobutyl ketone, tetrahydrofuran, 2,6-lutidine, ethyl acetate, isopropanol, chloroform, cyclohexanone, methyl ethyl ketone, methyl n-propyl ketone, 2-picoline, dioxane, ethanol, nitroethane, pyridine, acetone, methoxyethanol, acetic acid, acetonitrile, methanol, nitromethane, m-cresol; and/or water. Other polar solvents may be used.

The nonpolar solvent may be a liquid solvent having a polarity index of less than 4. In embodiments of the invention, the nonpolar solvent may be one or more of squalane, isooctane, n-decane, 1,1,2-trichlorotrifluoroethane, cyclohexane, n-hexane, pentane, cyclopentane, heptane, petroleum ether, carbon disulfide, n-butyl chloride, carbon tetrachloride, dibutyl ether, triethylamine, diisopropyl ether, toluene, o-xylene, p-xylene, methyl t-butyl ether, bromobenzene, chlorobenzene, iodobenzene, o-dichlorobenzene, diethyl ether, benzene, dichloromethane, ethyl bromide, fluorobenzene, ethylene dichloride, isopentanol, ethylene chloride, 2-propanol, n-butanol, n-propanol, and/or tert.-butanol. Other nonpolar solvents may be used.

The polar plant extracts, for example Extract 1B 30, may be an active ingredient in preservatives, antioxidants, cleansing agents, and disinfecting agents.

The nonpolar extracts, for example Extract 1A 28 and Tropolone Extract 1C 36, may be useful as components of preservative compositions (Hiroyasu Y., Takatoshi Y; Takako Y. (1998), Japanese Journal of Food Chemistry 5(2)), antifungal agents (Morita, Y; Matsumura, E., Tsujibo, H. et al. (2002), Biological and Pharmaceutical Bulletin, 25(8): 981-5; and Inamori, Y.; Morita, Y. (2001), Aroma Research 2(2): 137-143; and Grohs, B., Wegen, H W., and Kunz, B. Holz Als Roh-und Werstoff (1999), 57(4): 227-281), antioxidant compositions, fragrances, cleansing agents, antibacterial agents (Anderson, A B et al. Acta Chem. Scand (1948) 2:644; Erdtman, H. et al. Nature (1948),161:719; Gripenberg, J. Acta Chem. Scand. (1948) 2, 639, Arima, Y; Nakai Y; Hayakawa R. et al. (2003), J Antimicrob Chem 51(1):113-22, and Inamori, Y. Shinohara, S., Tusjibo, H. et al. (1999), Biological & Pharmaceutical Bulletin, 22(9): 990-3), disinfecting agents (Miyamoto D., Kusagaya Y I, Endo N. et al. (1998), Antiviral Research, 39(2): 89-100), insecticidal agents (Inamori, Y.; Morita, Y. (2001), Aroma Research 2(2): 137-143; Ahn-Young-Joon, Lee-Seong-Baek, Lee-Hoi-Seon et al. (1998), Journal of Chemical Ecology 24(1): 81-90), and/or anticancer agents (Masumura E., Morita Y, Date T et al. (2001), Biological & Pharmaceutical Bulletin, 24(3): 299-302 and Miyamoto, D., Endo, N., Oku, N. et al. (1998), Biological & Pharmaceutical Bulletin, 21(12):1258-62). The nonpolar extracts may further be used in the manufacture of fragrances, disinfecting and cleaning agents, antifungal agents, preservative agents, and toiletries such as toothpastes (Osawa K; Matsumoto T; Maruyama T. et al. (1990), Bulletin Tokyo Dental College 31(1): 17-21), shampoos, and soaps etc.

The nonpolar extracts may be used in the manufacture of medicaments for treating infection, cancer, fungal overgrowth, and parasite infestation in mammals, including humans.

The nonpolar extracts of the invention may be used as a starting point in the synthesis of other compounds, for example potent antitumour compounds such as described in Yamamoto M., Hasigaki K; Kokubu N., et al. (1984), J Med Chem 27(12): 1449-53; and in Yamamoto, M., Hashigaki, K; Ishikawa S. (1985), J Med Chem 28(2):1026-31.

Polar compounds such as flavanoids and diterpenes in Thuja orientalls have been shown to be useful as 5α-reductase inhibitors and useful in treating alopecia, controlling excess hair growth, and in treating acne (see, for example, Canadian patent application CA 2178528).

Methyl thujate may be used as an ingredient in fragrances, or as a fragrance in other products.

Solid plant materials after extraction 13 may be useful for the construction of hypoallergenic wood products including, but not limited to, particle board, artificial logs for home fireplaces, etc. In these plant materials 13, the plicatic acid component implicated in asthma causation may be greatly reduced. Indeed, these solid plant materials 13 may have certain extracted components reintroduced during manufacture, such as methyl thujate for fragrance, or other tropolones for preservation, but remain virtually free of plicatic acid. Thus a type of hypoallergenic cedar wood material is possible for construction.

The process according to one embodiment of the invention may include removing a proportion of the lignin from the nonpolar phase, Extract 1A 28, with an additional wash of nonpolar solvent, to form Tropolone Extract 1C and Lignin Extract 1D 38. The Lignin Extract 1D 38 may be useful in the manufacture of antioxidants agents, or as an antioxidant ingredient in various foods, beverages, and industrial mixtures.

See for example Canadian Patent 880830. Sep. 14, 1971 to Karchmar, A. & McDonald, K., and U.S. Pat. Nos. 3,644,481And 3,754,943.

The Tropolone Extract 1C 36 may be useful in the manufacture of medicaments for treating infection, disinfecting agents, fragrances, antibacterial agents, anticancer agents, antifungal agents, antiparasitic agents and insecticidal agents.

Extract 1B 30 may include many useful nonvolatile compounds including but not limited to: plicatic acid, plicatin, thujaplicatin, thujaplicatin methyl ether, dihydroxythujaplicatin, hydroxythujaplicatin methyl ether, dihydroxythujaplicatin methyl ether, plicatinaphthalene, plicatinaphthol, and/or gamma-thujaplicatene.

In some embodiments of the invention, Extract 1A 28 and Tropolone Extract 1C 36 contain a compound of the formula (1) 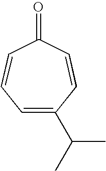
at high levels such as 15%.

This compound may be useful as a starting material in chemical synthesis, in antibacterial and disinfectant compositions, in antifungal, insecticidal, or preservative agents, etc.

The extracts and compounds of the invention may for example be formulated for topical use in creams ointments, tinctures, soaps or washes. The extracts of the invention may be useful as cancer treatments, enzyme inhibitors or pharmaceuticals, in the form of topicals, coatings, injectables, and the like.

Optionally, the extracts or compounds of the invention may be used as a preservative in food preparation in small quantities, to prevent the growth or survival of pathogenic agents. The compounds and extracts of the invention may be used in insecticidal, antifungal or anti-parasite formulations or treatments, and as an ingredient in cosmetics and health aids such as toothpaste, mouthwash, and hair treatments including shampoos, conditioners and rinses. The compounds and extracts may also be used as topical antiseptics or antifungals, or in formulations for insect repellents. They may be added to textiles and plastics as a disinfectant, conditioner, insect repellent and deodorant.

In some embodiments, the compounds and extracts may also find use as an ingredient in household products such as carpet shampoos, floor-cleaning agents, surface cleaning agents and polishes.

In some embodiments, the extracts may provide an economical starting material for the isolation or manufacture of medically useful terpenoids, flavanoids, or tropolones.

In some embodiments, the compounds and extracts may find use as intermediates in the manufacture of derivatives based on the chemical platform of the individual extracted compounds, a platform that is based on the unique seven sided ring structure of the tropolones molecule.

In a typical situation, plant materials such as those from Arizona Cypress (Cupressus arizonica); McNab Cypress, (Cupressus macnabiana); One Seeded Juniper, (Juniperus monosperma); Atlantic White Cedar (Chamaecyparis thyoides); Chamaecyparis obtusa (Kiso-Hinoki), Thujopsis dolabrata, Western Red Cedar (Thuja plicata Don.), and Northern White Cedar (Thuja occidentalis) may be harvested and optionally macerated to provide a high surface area to volume of plant tissues. This product will hereafter be referred to as “plant materials”, and will be used to refer to the solid materials subject to extraction, and “extracted plant materials” to the solid materials remaining after extraction. The starting plant materials may include bark, stem-wood, root wood, branch wood, foliage, fruits and seeds of the species used to prepare the plant materials. Plant materials may be from fresh or old plants or trees, and may be waste material from harvest or manufacture, including wood chips, sawdust, and stumps. One specific species of plant materials, or a number of species, may be used.

In methods according to an embodiment of the invention, plant materials containing desired compounds are treated with solvents to extract and separate desired substances from the plant tissue. Freshly harvested plant materials may be preferred, but old wood may also be used to produce products with a lower potential yield but at a lower materials cost.

The methods may involve the use of a polar solvent and a nonpolar solvent applied sequentially to extract and purify the desired compound mixtures. The extracts can then be concentrated, and may be either used as a mixture, or subjected to isolation of the various member compounds or groups of compounds.

The plant material, which may be macerated, is then mixed with enough of the polar solvent to extract compounds by dissolving them from the plant material. The reaction may take place in a container.

In FIG. 1, the plant material and solvent mixture is represented at 12. The container used may be small, for example a few hundred millilitres, but is more typically an industrial sized vat or tank of several litres to hundreds to even thousands of litres. The vat may be metal, non-reactive plastic such as polycarbonate, wood, glass, or a combination of those materials. The vat may be polymer (i.e. Teflon™) or glass lined. The process may be a continuous batch process or a single batch. The tanks or vats may be clustered such that the polar solvent will flow in a countercurrent fashion starting fresh in tanks of plant materials that have been previously processed and going on to tanks containing less processed, and finally unprocessed, plant materials.

Extraction may be effected by immersion of the plant materials in, and/or percolation through the plant materials by, the polar solvent. The mixture may be agitated, kept at a low atmospheric pressure, or at an elevated temperature to improve extraction.

In this way the polar solvent may be “loaded” with the maximum amount of the extractable compounds and those compounds remaining in the plant materials may be kept to a minimum.

The polar solvent may be one or more of the polar solvents as previously described, but is not limited thereto. Table 1 is a table of solvents showing a polarity index for each (Snyder 1974, 1978). In some embodiments, the polar solvent has a polarity index of at least 4. Table 2 lists categories for some of the solvents in Table 1.

TABLE 1
Solvents and Their Characteristics

| | Polarity | Water Solubility | Boiling Point |
|---|---|---|---|
| Solvent | Index | (grams/100 ml) | (° C.) |
| Squalane | −0.8 | | 285 at 25 mm Hg |
| Isooctane | −0.4 | Insoluble | 99.24 |
| n-Decane | −0.3 | 0.1 | 174.1 |
| 1,1,2- | 0.0 | 0.02 | 47.57 |
| Trichlorotrifluoroethane | | | |
| Cyclohexane | 0 | Insoluble | 80.72 |
| n-Hexane | 0 | 0.00947 | 68.7 |
| Pentane | 0.0 | 0.04 | 36.07 |
| Cyclopentane | 0.1 | Insoluble | 49.26 |
| Heptane | 0.1 | .01 | 98.43 |
| Petroleum Ether | 0.1 | N/A | 35-60 |
| Carbon disulfide (VIb) | 1 | 0.1185 | 46.2 |
| n-Butyl Chloride | 1.0 | .07 | 78.44 |
| Carbon tetrachloride | 1.7 | 0.08048 | 76.7 |
| (VIb) | | | |
| Dibutyl ether (I) | 1.7 | Insoluble | 141 |
| Triethylamine (I) | 1.8 | .02 | 88.9 |
| Diisopropyl ether (1) | 2.2 | .2 | 68.5 |
| Toluene (VIb) | 2.3 | .0526 | 110.62 |
| O/p-Xylene (Vib) | 2.4 | .0175 | 138.3 |
| Methyl t-Butyl Ether | 2.5 | 5.1 | 55.2 |
| Bromobenzene (VII) | 2.7 | 0.1 | 155 |
| Chlorobenzene (VII) | 2.7 | .0497 | 131.69 |
| Iodobenzene (VII) | 2.7 | N/A | 188 |
| o-Dichlorobenzene | 2.7 | N/A | 180.48 |
| Diethyl ether (I) | 2.9 | 6.9 | 34.6 |
| Benzene (VIb) | 3 | 0.18 | 80.1 |
| Dichloromethane | 3.1 | 1.32 | 39.75 |
| Ethyl bromide (VIa) | 3.1 | 0.1 | 38.4 |
| Fluorobenzene (VII) | 3.3 | 0.1-1 | 85.1 |
| Ethylene Dichloride | 3.5 | 0.8608 | 83.48 |
| Isopentanol (II) | 3.6 | 0.054 | 130 |
| Ethylene chloride (V) | 3.7 | 0.8608 | 83.5 |
| 2-propanol (II) | 3.9 | Miscible | 82.26 |
| n-Butanol (II) | 3.9 | 6.32 | 117.5 |
| n-Propanol (II) | 3.9 | N/A | 97.2 |
| Tert.-Butanol (II) | 3.9 | Miscible | 82.2 |
| 2-Methyl-1-propanol | 4.0 | 9.5 | 107.7 |
| Methyl Isoamyl | 4.0 | Slightly | 144.9 |
| Ketone (VIa) | | | |
| n-Butyl Acetate | 4.0 | 0.68 | 126.11 |
| Methyl Isobutyl | 4.2 | 1.9 | 117.4 |
| Ketone (VIa) | | | |
| Tetrahydrofuran (III) | 4.2 | 30 | 66 |
| 2,6-Lutidine (III) | 4.3 | N/A | N/A |
| Ethyl acetate (VIa) | 4.3 | 8 | 77.11 |
| Isopropanol (II) | 4.3 | Miscible | N/A |
| Chloroform (VIII) | 4.4 | 0.795 | 61.15 |
| Cyclohexanone (Via) | 4.5 | 5-10 | |
| Methyl ethyl | 4.5 | 25.6 | 79.64 |
| ketone (VIa) | | | |
| Methyl n-Propyl | 4.5 | 4.3 | 102.4 |
| Ketone (Via) | | | |
| 2-Picoline (III) | 4.8 | Miscible | 129.5 |
| Dioxane (VIa) | 4.8 | Miscible | 101.32 |
| Ethanol (II) | 5.2 | Miscible | 78.3 |
| Nitroethane (VIb) | 5.3 | 4.5 | 114 |
| Pyridine (III) | 5.3 | Miscible | 115.25 |
| Acetone (VIa) | 5.4 | Miscible | 56.2 |
| Methoxyethanol (IV) | 5.7 | Miscible | 124.6 |
| Acetic acid (IV) | 6.2 | Miscible | 117.9 |
| Acetonitrile (Via) | 6.2 | Miscible | 81.60 |
| Methanol (II) | 6.6 | Miscible | 64.7 |
| Nitromethane (VIb) | 6.8 | 9.5 | 101.1 |
| m-Cresol (VIII) | 7 | 1.932 | 88-94 |
| Water (VIII) | 9 | N/A | 100 |

TABLE 2
Classification of Most Solvents in TABLE 1 (Snyder)

| Group | Solvents |
|---|---|
| I | Aliphatic ethers, trialkyl amines, tetramethylguanidine |
| II | Aliphatic alcohols |
| III | Pyridines, tetrahydrofuran, amides (except the more acidic |
| | formamide) |
| IV | Glycols, glycol ethers, benzyl alcohol, formamide, acetic acid |
| V | Methylene chloride, ethylene chloride, tricresyl phosphate |
| VIa | Alkyl halides, ketones, esters, nitriles, sulfoxides, sulfones, |
| | aniline and dioxane |
| VIb | Nitro compounds, propylene carbonate, phenyl alkyl ethers, |
| | aromatic hydrocarbons |
| VII | Halobenzenes, diphenyl ether |
| VIII | Fluoroalkanols, m-cresol, chloroform, water |

When viewed for its solvent properties, water is effective at penetration of most plant tissue. However, water alone usually offers insufficient penetration of the dense, thick-walled woody plant materials used in the present application.

In some embodiments of the invention, when polar solvents other than water are applied to plant materials, the results are improved intracellular penetration of the plant materials over those obtained by the use of water as a solvent.

In some embodiments, the polar solvent may be soluble in water. The polar solvent may for example have a solubility in water of at least 70%, at least 80%, or at least 90%.

In some embodiments of the invention, the plant materials may be immersed in the polar solvent for a period of time, from a few minutes to several hours, until the desired amount of extraction from the plant materials has occurred. The extraction conditions in the processes may be maintained for an extraction period of from about one minute to three days, from about one to 24 hours; from about four to 12 hours; about four hours, about six hours, or about 12 hours. The period can be longer or shorter, depending on the required yield and the physical and chemical condition of the plant materials. These conditions may include reduced pressure and/or elevated temperature according to the plant materials, boiling points of the solvents being used, and desired composition of extracts.

Temperature and pressure of the solvent/plant materials mixtures can be adjusted to affect the rate at which the plant materials are extracted. Higher temperature and/or lower pressure will result in higher rates of extraction, but may also lower yields of unstable or heat-labile compounds.

After extraction, the extracted plant materials are separated from the solvent by physical means. This may be done using a sieve or series of sieves, filters, manual raking, netting of various sized holes, centrifugation, ultracentrifugation, or the use of any other device with openings adequate to let only the liquid (comprised of the polar solvent pregnant with dissolved plant extracts, as well as water from the plant materials), through while retaining the greater part of the extracted plant materials. The resulting solvent phase may be referred to as ‘pregnant’.

After extraction, the extracted plant materials 13 may for example be used for some other purpose such as in pulp and paper production, for alcohol manufacture, for biodiesel, for composting, for making structural elements like boards and sheets using adhesives, for example Portland 10 cement or formaldehyde glue, as fuel for cogeneration, returned to the forest as fertilizer, used as playground or gardening surface materials, or used as daily cover at landfills.

The pregnant liquid may then be further processed for example by filtering, centrifugation or by settling, to remove smaller particles of plant materials not removed by the first separation. In alternative embodiments, the filter used may for example have openings, of one micron, 5 microns, 10 microns or 100 microns. In alternative embodiments, the openings may be larger or smaller depending on the desired product specifications. A series of filters of decreasing pore size may also be used.

The extract resulting from these first steps can be called Extract 1 20 as shown in FIG. 1, and may be a product for use and sale in and of itself. Extract 1 20 contains both the volatile and nonvolatile plant materials extracts. It consists of the original polar solvent, extracted compounds that are dissolved in the solvent, and any residual water derived from the original plant materials.

Extract 1 20 may be sold or used as is, further processed as described below, or may be further processed by distillation 22 to produce a more concentrated solution. This distillation 22 will be done at low temperatures, preferably from about 15-80° C., and preferably at pressures less than atmospheric for example in the range 500-760 mmHg, to avoid any loss of the extracted compounds. The concentrated Extract 1 20 may thereby be reduced by from 10% to 90% of its original volume by this step, depending on the starting concentration, or degree of reduction of volume or increase of concentration required.

In an embodiment of the invention, a nonpolar solvent may be added to Extract 1 20 to form a polar/nonpolar mixture 24. The nonpolar solvent may be called an ‘opposing solvent’. In one example, the nonpolar solvent is dichloromethane, but other possible solvents are, for example, petroleum ether, benzene, diethyl ether, hexane and pentane (see Table 1 solvents with a polarity index of less than four).

Extract 1 20 and the nonpolar solvent may be mixed well 24, for example in a separation tank, and allowed to partition. This step may for example take from a few minutes to a few hours or days, one minute to three days, from about one to 24 hours; from about four to 12 hours; about four hours, about six hours, or about 12 hours, and may result in two or more layers of immiscible fluids. As previously described, these conditions may include reduced pressure and/or elevated temperature according to the plant materials, boiling points of the solvents chosen, and desired composition of extracts.

In some embodiments, the polar solvent and the nonpolar solvent may be selected to be substantially immiscible. Polar and nonpolar solvents may accordingly be selected so that the solvents will separate in separate phases after the components are mixed together. The solubility of each solvent in the other may for example be less than 20%, 10%, 5%, 2%, 1%, or 0.1%.

The volatile components of Extract 1 20 may migrate to form a layer with the nonpolar solvent to form nonpolar Extract 1A 28 as shown in FIG. 1. The polar solvent, water, and the nonvolatile components of Extract 1 20 may also form a second layer, polar “Extract 1B” 30. The order of layering (upper or lower) may depend on the relative weights of first and nonpolar solvents used.

The layers constituting Extract 1A 28 and Extract 1B 30 may be separated by methods known in the art of liquid separations, for example by using a separation vessel with variously placed spouts, by siphoning, by pouring off the upper layer into another vessel, etc.

Once Extract 1A 28 is separated, the nonpolar solvent may be allowed to evaporate from it under temperatures and/or atmospheric pressure sufficient to result in evaporation, but not so high as to cause the volatile components of Extra 1A 28 to deteriorate. The boiling points in Table 1 provide an indication of which solvents will evaporate easily under various conditions.

In some embodiments, Extract 1A 28 may be concentrated or even desiccated and then may be extracted with another nonpolar solvent 34 to extract the tropolone type compounds into the nonpolar solvent to form “Tropolone Extract 1C” 36. The remaining material may be re-suspended in any of a number of solvents and may form “Lignin Extract 1D” 38, which may comprise the greater part of the lignin type compounds that were present in Extract 1A 28.

In some embodiments, the nonpolar solvent may be removed from Extract 1A 28 by distillation or similar means previously discussed, and the recovered solvent may be discarded, or more preferably, set aside for reuse in another cycle. The polar solvent may similarly be removed from polar Extract 1B 30.

Extract 1A 28, Extract 1B 30, Tropolone Extract 1C 36 and Lignin Extract 1D 38 described above may be further refined to separate and purify the volatile and nonvolatile components they respectively contain. Methods for refinement include, but are not limited to, crystallization, fractional distillation, gas chromatography, gas-liquid chromatography, high pressure liquid chromatography, thin layer chromatography and other forms of chromatography known to those skilled in the art. The refined extracts can then be used or sold as mixtures, or in more purified forms.

In some embodiments, refined Extracts 1A 28, 1B 30, 1C 36, and 1D 38 may also be further modified or derived to form desired products, or act as intermediates for manufacture of other compounds for industrial or medical applications.

In some embodiments of the invention, a relatively large amount of nezukone has been isolated from Extract 1A 28 and Extract 1C 30. Nezukone is a seven-sided ring tropolone structure present at about 15% by weight of the volatile fraction of the extractives of one embodiment of the invention.

Purified Extract 1A 28 or Extract 1C 36 or derivatives thereof find use as an antimicrobial against various species of bacteria, including MRSA. Other species of bacteria that may be impeded or eradicated by Extract 1A 28 of the invention include Streptococcus spp., Pseudomonas spp., Enterococcus spp., Candida spp., Cryptococcus spp., and Escherichia coli spp., Serratia spp., Proteus spp., Enterobacter spp., Klebsiella spp., Pseudomonas spp., as well as other sources of nosocomial infection. The products of the invention are useful in preventing the growth of species of pathogens that are resistant to traditional antibiotics such as vancomycin (i.e. vancomycin-resistant enterococci).

Extract 1A 28 or Extract 1C 36 also finds use as the basis for fragrance manufacture, research and discovery, and as an ingredient in antibacterial, anti-insecticidal, and antifungal preparations for use in hospital settings, food preparation settings, residential and industrial environments, and as lumber treatments. The components of Extract 1A 28 may be used as a platform for combinatorial chemistry in pharmaceutical and industrial chemical research and development. Extract 1A 28 may be used as is or further refined or purified prior to use, according to the application. Hospital and food environments will require more refined mixtures than industrial applications like insecticide and fungicide preparation.

Extract 1A 28 may be sold as bulk liquid at various concentrations to industrial users and manufacturers, or may be desiccated in whole or in part to crystalline material for easier shipping and greater stability.

As prepared by the processes of the invention, Extract 1B 30 contains a number of useful nonvolatile components such as plicatic acid, plicatin, thujaplicatin, thujaplicatin methyl ether, dihydroxythujaplicatin, hydroxythujaplicatin methyl ether, dihydroxythujaplicatin methyl ether, plicatinaphthalene, plicatinaphthol, pigmaeine and iso-pigmaeine, and gamma—thujaplicatene. Extract 1B 30 or its components are sold to chemical reagent companies as a starting material for combinatorial chemistry, or as a research tool for agricultural and pharmaceutical sciences. Plicatic acid may also be used as a preservative and an antioxidant.

The chemical structures of some of these components are shown below.

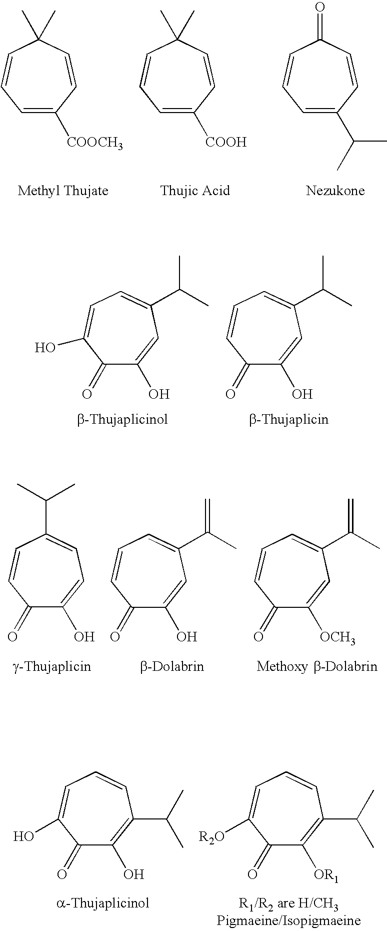

Lignin Extract 1D 38 contains lignin type compounds, which find use as antioxidants for cosmetics, industrial applications, and articles of manufacture.

While specific embodiments of the invention have been described and illustrated, such embodiments should be considered illustrative of the invention only and not as limiting the invention as construed in accordance with the accompanying claims. Any cited patents, patent applications, and published referenced are hereby incorporated by reference in those jurisdictions permitting such incorporation.

### EXAMPLES

### Example 1

### Extraction Process

The extraction procedures given in the following section of Example 1 describes a small commercial scale extraction conducted in an explosion proof facility, using trained staff and explosion-proof apparatus dedicated to that purpose.

Approximately 300 litres of run-of-mill western red cedar tissues freshly macerated in a commercial flail shredder were loaded into a Littleford model FKM-600-D-2Z stainless steel tank of 600 litre capacity. Tank doors were sealed shut and fresh commercial grade methyl alcohol (MA) (methyl alcohol, CH₃OH, supplied as 99% pure commercial grade by Univar Canada Ltd.) was added through an inlet valve in sufficient quantity to cover the plant materials. An agitator built into the inside of the tank was used to stir the mixture for two minutes. The mixture was allowed to interact at 30° C. and 760 mm Hg pressure for about 12 hours.

The MA was then allowed to drain away under gravity via a drain valve at the base of the tank. At the conclusion of draining, when free flow had ceased but drips were still occurring, the valve was closed. Approximately 200 litres of fresh MA were then introduced through a valve at the top of the tank to the plant materials. Using the internal paddle in the tank, the plant materials were agitated for five minutes. When the agitator was turned off, the inlet valve for the alcohol was closed and the drainage valve at the base was opened. The ‘pregnant’ MA was again drained into the same stainless steel holding tank used to store the first pregnant MA extract drained from the Liftleford tank.

The ‘pregnant’ MA solution was then filtered to remove wood debris and wood fines (extracted plant materials) down to 1.0 micron diameter by pumping from the holding tank through a 4.1 litre Pressure Leaf Filter; Type 116.334, Series 36-1.5-28, Model 1-036 made by Industrial Filter and Pumps, Cicero, Ill. The resultant filtered polar solvent solution containing MA, water originating from the plant materials, and dissolved extracts from the cedar plant materials, was labeled Extract 1 and stored in a stainless separation tank for the next stage of processing.

Solvent was removed from the extracted plant materials in a Littleford tank/dryer. A vacuum of between 200 and 700 mm Hg was exerted on the tank/dryer and the internal agitator set at a rotation speed of between 5-10 rotations per minute. Into the slowly agitating plant materials, steam was introduced. Low pressure steam leaving the boilers at 83-104 kPa, was delivered at a rate of 3 kg/hr, heating the plant materials to 90° C. This temperature was maintained for 6 hours. Solvent vapors driven from the wood were drawn from the reactor through a manually operated vapor port at the top of the apparatus, condensed, collected and added to Extract 1.

Extract 1 was concentrated using a Contherm brand Model 6x2 scraped surface jacketed evaporator manufactured by De Laval, Newbury, Mass. Solvent was evaporated using a water jacket set in the range of 65-85° C. Absolute pressure in the system was kept at 22-23 mm Hg. The temperature of the concentrated MA leaving the evaporator was in the range 27-35° C. The MA volume in Extract 1 was concentrated to about 12-16% of the original volume.

A separation tank constructed of stainless steel with a conical bottom was then used. In the apex of the cone a sight glass was attached, and a drain valve was attached to the lower end of the sight glass. For mixing, an electric driven impeller was inserted downwards into the tank through an aperture made in the tank lid. The tank lid was sealed with a gasket. With the impeller installed, the aperture in the lid for the shaft was sealed with a gasket.

To a concentrated solution of Extract 1 in the separation tank, nonpolar solvent dichloromethane (DCM) (CH₂CL₂, supplied as 99% pure, commercial grade by Univar Canada Ltd.), was added. The resulting mixture was mixed thoroughly by the impellor and allowed to still (stop moving and settle out) and to partition for one hour. DCM and the volatile components of the Extract 1 formed a lower non-aqueous phase or layer (Extract 1A). MA, water and the nonvolatile components of the extract formed an upper aqueous phase (Extract 1B).

The upper and lower layers were separated manually by opening the valve at the base of the sight glass on the separation tank and allowing the DCM to flow out under gravity into a clean empty stainless steel container. When the partition horizon separating the lower DCM layer from the upper MA layer appeared in the sight glass of the emptying separation tank, the valve was partially closed to slow the flow. When the partition horizon arrived at the slightly open valve, the valve was completely closed. The tank containing the DCM was taken from under the separation tank and stored. It was replaced under the separation tank valve by a second clean empty stainless steel tank. The valve on the sight glass was opened allowing the alcohol layer to completely drain into the second storage tank. The separation of the layers was then complete.

Excess MA and water were removed from Extract 1B by distillation at 105° C. to dryness, leaving the non-volatile fraction of the extract as a dry powder. This non-volatile fraction made up about seven percent by weight of the original plant materials, and was stored in brown glass containers.

Extract 1A was concentrated in batches using a Buchi Rotovaporator™ Model R-153. The water bath was set at a temperature of 20° C. with a partial pressure of approximately 300 mm Hg established by a vacuum pump across the solution. DCM was allowed to distill off to be collected for reuse in another cycle. As the recovery of DCM reached approximately 98% of the amount at the start of evaporation, an excess of anhydrous ethyl alcohol was added to the flask (anhydrous ethyl alcohol, formula C₂H₅OH, 100% pure laboratory grade). Distillation was continued at 20° C. until the remaining 2% of MA was removed. The volatile fraction of the wood extract dissolved in the excess ethyl alcohol is a purified Extract 1A. Alternatively, the 2% remaining MA is carefully distilled off as before to leave a sticky dark brown colored solid in the retort. In this case, the solid is the Purified Solid Extract 1A.

In an alternative procedure, DCM was evaporated from Extract 1A at low temperatures. The residue was then further extracted with ethyl ether to remove the tropolones into a Tropolone Extract 1C, leaving the lignin type compounds in a Lignin Extract 1D. TLC procedure used after the cleanup was a silica gel plate UV254 with the solvent system ethyl acetate:hexane in a 4:6 ratio.

Purified Extract 1A is about 6% by weight (yield) of the original plant materials and contains over one hundred and twenty-five compounds that have been identified by gas chromatography mass spectrometry (GCMS). Most of these compounds are in trace quantities and some have been identified as follows:

TABLE 3
Some of the Volatile Components Found in Thuja plicata Don.

| Compound | Relative Amount | Application |
|---|---|---|
| Methyl thujate | 6-8% | Fragrance |
| Thujic Acid | 24-28% | Antiseptic/insect repellent |
| Alpha thujaplicin | 0-1% | Antibiotic/phytogrowth |
| | | inhibitor |
| Beta thujaplicin | 4-5% | Antibiotic/phytogrowth |
| | | inhibitor |
| Gamma thujaplicin | 10-12% | Antibiotic/phytogrowth |
| | | inhibitor |
| Dolabrin | 0-1% | Antibiotic/phytogrowth |
| | | inhibitor |
| Nezukone | 15% | Antiseptic |
| Carvacol | 0-1% | Insect repellent |

### Example 2

### Analysis of Extracts

Gas chromatography was carried out on Extract 1A to identify the volatile compounds derived from Thuja plicata Don. The method of running the sample was as follows: The run time used was 33 minutes. An Agilent Technologies 6890N Network GC System gas chromatography device was used. The Carry Gas was Helium and the injection volume was 1 μl.

Results of the GC are shown in Table 4.

The results showed a number of peaks at 16 and 24 minutes some of which are known compounds methyl thujate, thujic acid, beta thujaplicin, and gamma thujaplicin.

Readings were done on batches of Extract 1A that had been processed in the initial plant materials extraction for various amounts of time, and the results showed a time dependent increase in the relative amounts of volatile compounds extracted. The data are shown below. Two injections of 2 μl each were run and the results averaged below.

TABLE 4
Average Peak Areas for Extract GC

| | Methyl | | | |
|---|---|---|---|---|
| Batch | thujate | Thujic acid | B-thujaplicin | Λ-thujaplicin |
| 20 minutes | 505 | 424 | <DL | <DL |
| 40 minutes | 1495 | 1019 | <DL | 320 |
| 150 minutes | 2228 | 1576 | <DL | 461 |
| 12 hours | 2436 | 1812 | 175 | 638 |

TLC was also used to identify the separation and purification of compounds throughout the process. HPLC was used to analyze the compounds.

Structural identification and qualitative/quantitative determinations of individual and/or group of tropolone(s) are carried out using a combination of following analytical methods, some as described in the literature; Thin Layer Chromatography (TLC), Capillary Gas Chromatography (GC), and Gas Chromatography-Mass Spectrometry (GC-MS). The HPLC method was used for these studies.

All organic solvents were analytical grade, and HPLC grade solvents were used for sample preparations involving HPLC and GC analysis. Water used was RO grade, or HPLC grade for HPLC mobile phases.

### Example 3

### Comparison of Extraction Methods

Extraction methods were compared to determine the relative compositions and efficiency of yields.

a. Extraction with Water.

A sample of cedar wood was placed in a container of water and heated to 95° C. The sample was allowed to soak for 1-6 hours. The aqueous phase was recovered by filtration and the ‘spent’ extracted plant materials were discarded. A sample of the aqueous phase was taken for analysis of its composition. Results are shown in Table 5.

b. Extraction with Steam.

A sample of wood was placed in a metal retort and heated with ‘dry’ steam delivered at temperatures ranging from 150-190° C. at absolute pressure of between 96.5 kPa and 193 kPa for a period of 1-6 hours. The hot vapours exiting the retort were condensed in a water-cooled heat exchanger running at from 6-26° C. at atmospheric pressure. Separation of the extract from the water was made using density differences between the water insoluble extract having a density greater than 1.0 at temperatures less than 10° C., and the water with a density of 1.0. A sample of the extract was taken for analysis, and results shown in Table 5.

c. Extraction with Two Solvents.

A sample of cedar wood was placed in a container to which sufficient MA was added to keep the upper surface of the wood wetted. The mixture was allowed leach or soak with periodic stirring, for a period of from about 2-12 hours at temperatures ranging from 15-30° C. The alcohol was then separated from the wood and an equal volume of DCM (to the MA) was added. This mixture was allowed to stand for a period ranging from 1 to 4 hours at 10-25° C. at atmospheric pressure. When fully partitioned the upper and lower solvents were separated by density differences as earlier described.

The DCM/Extract 1A mixture was separated by distillation of the DCM, which was recovered. Remaining in the retort was the volatile fraction of the wood extract. A sample of this extract was taken for analysis of its composition.

The MA/Extract 1B mixture was also separated by distillation of the MA, which was recovered.

The compositions of the major components of the extract were analyzed by gas chromatography. Results shown in Table 5 demonstrate that the extract produced by the solvent method was higher in thujic acid, the thujiplicins, and in plicatic acid than in the water and steam extracts.

d. Extraction of Cedar Sawdust Using Two Solvents.

Method using MA and DCM

A sample of fresh western red cedar sawdust was collected at a sawmill. A portion of the sample was placed in a Soxhelet apparatus, and MA was added in excess. The apparatus was run for 6 to 12 hours. On completion, the MA was separated from the extracted plant materials, and the extracted plant materials discarded. To the MA fraction was added an equal volume of DCM. The solution was mixed thoroughly for between about 5 and 60 minutes. Upon cessation of mixing, the mixture of solvents partitioned and the layers were separated as earlier described. The MA fraction was stored and the DCM solution sampled for analysis of its contents by Gas chromatography. Results are shown in Table 5.

Method Using Acetone and DCM

The method using MA and DCM was performed using acetone instead of MA. The acetone fraction was stored and the DCM fraction were sampled for analysis of its contents by gas chromatography. Results are shown in Table 5.

e. Extraction of Cedar “Hog Fuel” Using Two Solvents.

A sample of freshly processed western red cedar wood industrial waste, consisting of shredded waste wood (called “hog fuel” in the vernacular of the wood milling industry), was sampled. The above method for cedar sawdust extraction using MA was followed. The MA fraction was stored and the DCM sample was sampled for analysis of its contents by gas chromatography. This test was repeated three times with fresh batches of the same wood sample. All three extractions were conducted under the same experimental conditions. Average values from three runs are shown in Table 5.

TABLE 5
Comparison of Various Extraction Methods and Materials

| | Solvents: | | | | | |
|---|---|---|---|---|---|---|
| | | | Acetone/ | MA/ | MA/ | MA/ |
| | Water | Steam | DCM | DCM | DCM | DCM |
| | Plant Materials: | | | | | |
| | | | | | | Hog |
| | Wood | Wood | Sawdust | Wood | Sawdust | fuel |
| Methyl | 0.5 | 55.0 | 4.6 | 8.0 | 3.1 | 6.8 |
| Thujate | | | | | | |
| Thujic Acid | 3.0 | 4.0 | 9.1 | 26.0 | 10.0 | 10.0 |
| Beta | 1.7 | <1.0 | 6.0 | 5.0 | 6.7 | 4.8 |
| Thujaplicin | | | | | | |
| Gamma | 10.1 | 0.0 | 13.0 | 12.0 | 14.3 | 8.3 |
| Thujaplicin | | | | | | |
| Plicatic Acid | 0.0 | 0.0 | Not | 39.7 | Not | Not |
| | | | present | | present | present |

All figures shown as percent by weight (% w/w)

### Example 4

### In Vitro study of Extract 1A Antibiotic Effects

The object of this study was to determine extract activity against methicillin-resistant S. aureus (MRSA), vancoymicin-resistant enterococci (VRE) and other species.

Method

An amount of 100 mg of Extract 1A was dissolved in about 0.5 mL DMSO, then further diluted in sterile distilled water (SDW) to make a 10,000 mg/L stock solution. This was used to make agar plates (Oxoid Isosensitest™ agar, pH 7.2 from Oxoid, Basingstoke, UK, supplemented with 10% sheep blood) containing Extract 1A at the following concentrations 0.1 mg/L, 1 mg/L, 10 mg/L, 0.1 g/L and 1.0 g/L.

Antimicrobial activity was measured by a standard agar-plate dilution procedure. The organisms were grown overnight in trypticase soy broth to yield approximately 109 colony forming units (CFU) per mL. The inoculum used was 104 cfu/spot, obtained by transferring 1 μL of a 1:100 dilution of the overnight culture to the plates with a Denley™ multiple inoculation device (Denley Instruments Ltd, Billingshurst, UK). Plates were incubated at 35° C. in air for 18 hours, with the exception of Cryptococcus spp., which was incubated for 48 hours. The minimum inhibitory concentration (MIC) was defined as the milligram of compounds per litre of medium at which there was a 99.9% or greater reduction in the original inoculum.

TABLE 6
Minimum Inhibitory Concentration of Extract 1A

| Number of | | MIC in |
|---|---|---|
| Plates | Organism | Grams |
| 5 | Enterococcus spp. | >1 |
| 5 | Vancomycin resistant | >1 |
| | Enterococcus spp | |
| 5 | Methicillin sensitive S. aureus | 1 |
| 5 | Methicillin resistant S. aureus | 1 |
| 5 | E. coli | 1 |
| 5 | P. aeruginosa | >1 |
| 5 | Candida albicans | 1 |
| 5 | Cryptococcus neoformans | 1 |

Extract 1A inhibited all 5 isolates of E. coli, all 10 isolates of S. aureus, all 5 isolates of C. albicans, and all 5 isolates of C. neoformans at 1000 mg/L. The actual MIC for E. coli, S. aureus and the yeast isolates is between 100 mg/L and 1000 mg/L as no plates were done between 100 mg/L and 1000 mg/L.

### Example 5

### Extraction and Separation of Hinokitiol

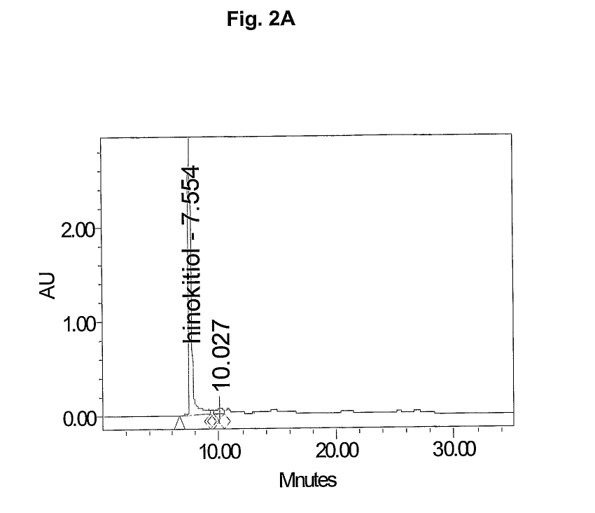

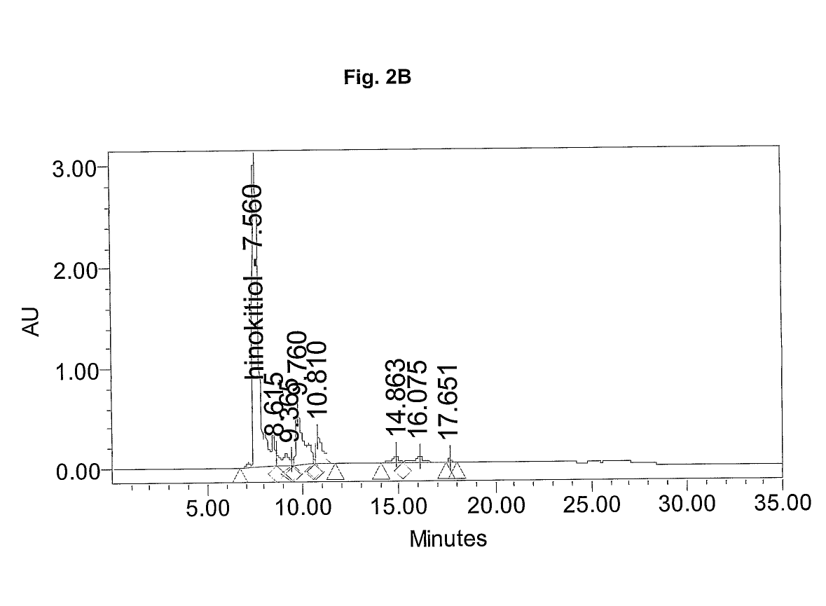

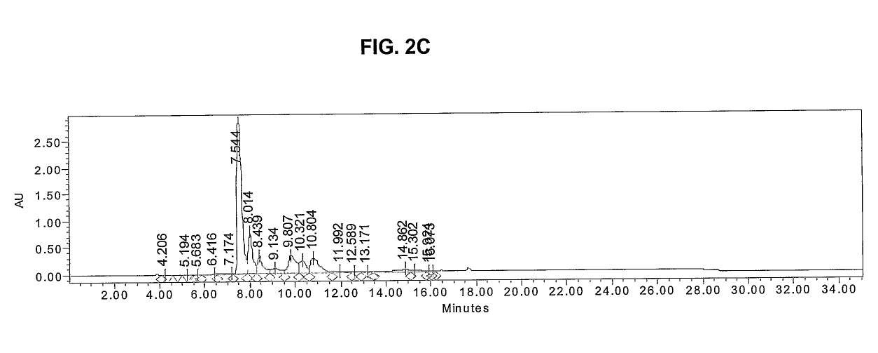

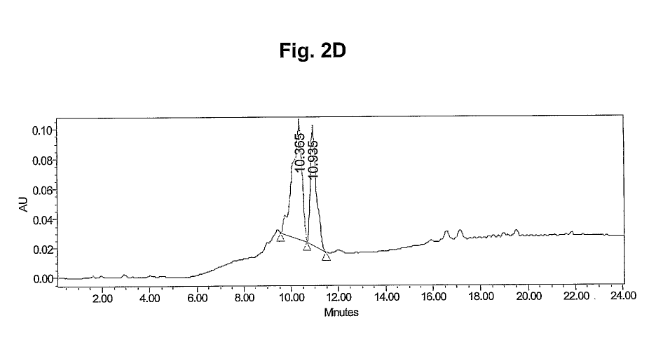

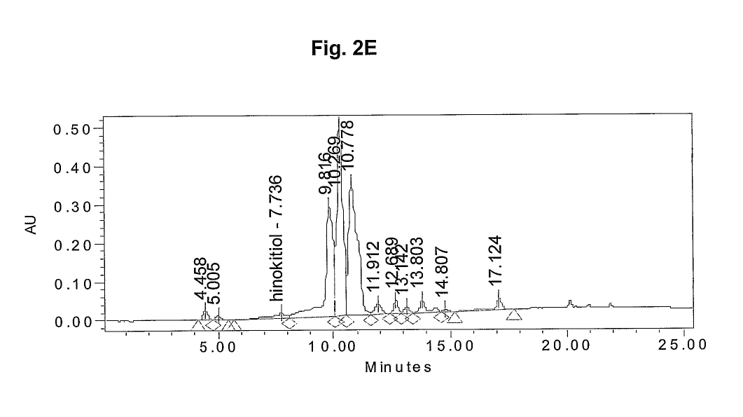

Red cedar (wood) (600 g) was extracted using three 24 h passes of methanol at room temperature. The extractions were combined and evaporated on the Rotavap™ to obtain about 60 g of dry extract. This was re-dissolved in water to form a suspension, and then was extracted with 5 passes of DCM to obtain a DCM extract of 15 g. The DCM extract was chromatographed over Silicon gel (200-400 mesh), then eluted with a hexane-acetone gradient solvent system. A total of 60 fractions, each 60 mL, were collected. Each fraction was developed on TLC plates and pooled according to their similarity in R_{f} values to get 34 fractions. Results for various fractions are shown in FIGS. 2A-E, which are HPLC traces showing relative levels of the extract components.

Fraction 7 was further separated using column chromatography over RP-18 silica gel eluted with methanol/water to get 15 fractions, of which fractions 2-6 contain hinokitiol. (as shown by TLC). Preparatory-TLC and HPLC were used for the further purification of about 5 mg of hinokitiol and about 20 mg thujic acid.

Larger Scale Extraction

Methods

NMR spectra were run on a Bruker Advance-40™ MHz spectrometer. EIMS were recorded on a Kratos™ MS 50 mass spectrometer. Silica gel (Merck, 200-400 mesh) was used for column chromatography. Thin-layer chromatography analysis was carried out on silica gel GF254™ plates (Merck) and PE SIL G plates (Whatman). HPLC was conducted using a Waters™ 518 pump combined with 4.6×75 mm waters symmetry C18 column, Waters™ 996 photodiode array detector, and a Waters™ 717 autosampler.

The dried bark of red cedar (4 Kg) was extracted with 3 passes of hot methanol, each 12 L, and the solutions were combined and concentrated in vacuo to obtain 400 g of residue. The methanol extract was dissolved in water, which was fractionated by liquid-liquid partition with DCM (5 passes, each 500 mL), and n-butanol (5 times, each 500 mL) to yield a DCM soluble portion of 60 g, and an n-butanol soluble portion 200 g, respectively. The combined DCM extract (60 g) was chromatographed over Silica gel (230-400 mesh), and eluted with hexane-acetone in a gradient solvent system. Fractions with similar R_{f} values by TLC were combined to give 50 fractions.

Fractions 3-10 were found to contain β-thujaplicin by TLC analysis (confirmed using a purchased standard, and as detected by color reagents). Fractions 3-10 were further separated using a column of silica gel (mesh 230-400) eluted with a hexane-acetone gradient to obtain 48 fractions. Of those fractions, fractions 8-13 contained thujic acid and fraction 15-30 contained hinokitiol.

The samples were filtered through a 0.2 micron filter into 1 ml injection vials, and injected onto the column after the column was equilibrated in methanol/water mobile phase for 12 min. Gradients were used in the HPLC run.

Hinokitiol was found to be present in the fractions by comparison of the UV spectra and retention times with those of the purchased controls, as well as in the HPLC analyses obtained by the spiking of samples (mixed with the methanol solution with 0.5 mg/mL of standards at the ratio of 1:1).

The percentage of hinokitiol in the fractions is shown in Table 7.

TABLE 7
Results of HPLC analysis

| | 1 | | 3 | | |
|---|---|---|---|---|---|
| | Methyl | 2 | Thujic acid | 4 | 5 |
| | thujate | Unidentified | Derivativess | Hinokitiol | unidentified |
| Fr. 2 | Methyl | | | | |
| | thujate | | | | |
| Fr. 3 | Methyl | | | | |
| | thujate | | | | |
| Fr. 4 | | 40 mg | | | |
| Fr 5 | | 10 mg | 50% | | |
| Fr. 6 | | 5 mg | | | |
| Fr. 9 | | | >80% | ˜10% | |
| Fr. 14 | | | 10% | ˜60% | |
| Fr. 18 | | | | ˜80% | |
| Fr. 22 | | | | ˜85% | |
| Fr. 24 | | | | ˜80% | |
| Fr 28 | | | | ˜50% | 50% |
| Fr. 31 | | | | ˜20% | 70% |
| Fr. 34 | | | | ˜10% | 70% |

The weight in Table 7 is the amount of the pure compound obtained from the related fraction. The percentage is from the HPLC analysis.

TLC and HPLC analysis indicate that fractions 9-34 contain hinokitiol. Figures See the HPLC trace of pure hinokitiol and the HPLC traces of fraction 14,18, and 22.

Thujic acid was obtained as co-crystal with another compound, which maybe the derivative of thujic acid. See the HPLC trace of thujic acid and those of fraction 5 and 9.

### Example 6

### Antimicrobial Effects of the Fractions

Hinokitiol standards and the pure compounds obtained from the column separation as well as the fractions in which β-thujaplicin exist in different concentration were tested for their anti-microbial activities.

Microorganisms: Laboratory strains of bacteria and fungus were obtained from Dr. Neil Towers' and Dr. Jovel's lab, The University of British Columbia. Seven species of bacteria and one species of fungus will be used in the screening process. The bacteria strains consisted of Staphylococcus aureus, methicillin resistant Staphylococcus aureus, Bacillus subtilis and Enterococcus faecalis, Escherichia coli, Pseudomonas aeruginosa, and Salmonella typhimurium. The fungal species was Candida albicans.

Methods: Disk diffusion assay on agar plates inoculated with the organisms being studies. The plates were divided into quadrants or sixths using a black marking pen. In quadrant 1 and 2, negative and positive controls were run (no treatment and gentamicin). Test compounds were run on the remaining available portions. The results are shown in Table 8

TABLE 8
Anti-microbial activities of pure compounds and fractions

| | | S. a. | | | | | | |
|---|---|---|---|---|---|---|---|---|
| | S. a. | MR | Bs | E. coli | E. f. | P. a | S. t. | C. a |
| Hinokitiol | +++ | +++ | +++ | ++ | +++ | + | +++ | +++ |
| standard | | | | | | | | |
| Thujic acid | ++ | ++ | ++ | ++ | ++ | — | + | ++ |
| standard | | | | | | | | |
| 4A (P) | — | — | — | — | — | — | — | — |
| 4 (P) | — | — | — | — | — | — | — | — |
| Fr. 2 | — | — | — | — | — | — | — | — |
| Fr. 3 | + | + | + | + | + | — | — | + |
| F. 5 | ++ | ++ | ++ | + | ++ | + | + | ++ |
| Fr. 9 | +++ | +++ | +++ | ++ | ++ | + | ++ | +++ |
| Fr. 14 | +++ | ++ | +++ | +++ | +++ | + | +++ | +++ |
| Fr. 15 | +++ | ++ | +++ | +++ | +++ | + | +++ | +++ |
| Fr. 18 | +++ | ++ | +++ | +++ | +++ | + | +++ | +++ |
| Fr. 20 | +++ | ++ | +++ | +++ | +++ | + | +++ | +++ |
| Fr. 24 | +++ | ++ | +++ | +++ | +++ | + | +++ | +++ |
| Fr. 28 | +++ | ++ | +++ | +++ | +++ | + | +++ | +++ |
| Fr. 31 | +++ | +++ | +++ | +++ | +++ | + | +++ | +++ |
| Fr. 34 | +++ | +++ | +++ | +++ | +++ | + | +++ | +++ |
| Gentamicin | +++ | +++ | +++ | +++ | + | +++ | +++ | — |

“+” active, “—” not active
4A (P) and 4 (P) are other compounds isolated in the purification process, not hinokitiol or thujic acid

Hinokitiol control, as well as the hinokitiol-containing fractions, demonstrated activities against all the bacteria and fungus used in above biological screening. The standard control, gentamicin was active against all the tested bacteria but not against C. albicans. Thujic acid crystals showed activities against some bacteria and fungus, however, its inhibition is weaker than hinokitiol.

In the TLC analysis on the fractions from the second column separation, Fractions 9-34 were found to contain β-thujaplicin. All those fractions displayed anti microbial activities.

The purity of hinokitiol did not need to exceed 98% to achieve good efficacy. The mixtures of all thujaplicin derivatives as prepared by the methods of the invention are useful, and the cost for separating all thujaplicin derivatives is significantly reduced, as well as the use of solvents which must be removed prior to application.

### Example 7

### Antioxidant Effects

Compounds in Extract 1B and Lignin Extract 1D of the invention are used to prevent oxidation in certain fats and oils. Fish oils, animal oils and vegetable oils are manufactured in the usual manner, and filter sterilized Extract 1b and/or Lignin Extract 1D is added after any heat processing. The extracts may be added to the oils either at the manufacturing stage or during consumer packaging. Nonpolar Extract 1A and Extract 1C, particularly alpha and beta thujaplicanol components, are also used to prevent oxidation in foodstuffs, including oils.

### Example 8

### Use of Plicatic Acid in the Manufacture of Plastics

The polar extract 1B and/or 1D are used in the formation of plastic. The purified plicatic acid polymerizes quickly under some conditions to form a black solid. As an additive to a known plastic forming agent, it changes the properties and strengths of the resulting composite. It is used to form a bio-plastic for use in various types of packaging

1. A process for preparing extracts from solid plant materials, wherein the plant materials comprise tropolones, lignins and polar molecules, the process comprising:
a) mixing the plant materials with a liquid polar solvent to form an extraction mixture;
b) maintaining said extraction mixture under extraction conditions effective to extract a proportion of the lignins, a proportion of the polar molecules and at least 50% of the tropolones in the plant materials into said polar solvent to form a pregnant polar solvent liquid phase, and a solid phase of extracted plant materials in said extraction mixture;
c) separating said pregnant polar solvent liquid phase from said solid plant materials;
d) mixing said pregnant polar solvent liquid phase with a substantially immiscible nonpolar solvent under partition conditions effective to partition the tropolones and lignins substantially into said nonpolar solvent and to partition the polar molecules substantially into said polar solvent, to form a partitioned nonpolar solvent phase comprising lignins and tropolones, and a partitioned polar solvent phase comprising the polar molecules; and
e) separating said partitioned polar solvent phase from said partitioned nonpolar solvent phase to obtain a polar plant extract and a nonpolar plant extract.

2. A process for preparing extracts from solid plant materials, wherein the plant materials comprise tropolones, lignins and plicatic acid, the process comprising:
a) mixing the plant materials with a liquid polar solvent to form an extraction mixture;
b) maintaining said extraction mixture under extraction conditions effective to extract a proportion of the plicatic acid, a proportion of the lignins and at least 50% of the tropolones in the plant materials into said polar solvent to form a pregnant polar solvent liquid phase, and a solid phase of extracted plant materials in said extraction mixture;
c) separating said pregnant polar solvent liquid phase from said solid plant materials;
d) mixing said pregnant polar solvent liquid phase with a substantially immiscible nonpolar solvent under partition conditions effective to partition the tropolones and the lignins substantially into said nonpolar solvent and to partition the plicatic acid substantially into said polar solvent, to form a partitioned nonpolar solvent phase comprising lignins and tropolones, and a partitioned polar solvent phase comprising plicatic acid; and
e) separating said partitioned polar solvent phase from said partitioned nonpolar solvent phase to obtain a polar plant extract and a nonpolar plant extract.

3. The process of claim 1 further comprising concentrating the partitioned polar solvent phase and the partitioned nonpolar solvent phase by removing the polar and the nonpolar solvents respectively to form a concentrated polar phase and a concentrated nonpolar phase.

4. The process of claim 3 wherein said polar solvent and said nonpolar solvent are removed by distillation.

5. The process of claim 3 wherein said polar solvent and said nonpolar solvent are removed using solid phase separation.

6. The process of claim 3 further comprising treating said concentrated nonpolar plant extract with an additional wash of nonpolar solvent effective to partition lignins and tropolones into a lignin extract and a tropolone extract.

7. The process of claim 6 wherein said additional wash of nonpolar solvent is comprised of diethyl ether.

8. The process of claim 1 wherein said solid plant materials are derived from a plant species selected from the plant order Cupressales.

9. The process of claim 8 wherein said plant species is selected from the group consisting of: Thuja plicata Don., Cupressus arizonica, Cupressus macnabiana, Juniperus monosperma, Chamaecyparis thyoides, Thujopsis dolabrata var. hondae and Thuja occidentalis.

10. The process of claim 9 wherein said plant species is Thuja plicata Don.

11. The process of claim 10 wherein the solid plant materials are derived from the trunk or branches of said plant species.

12. The process of claim 1 wherein said polar solvent is a liquid solvent having a polarity index of at least 4.

13. The process of claim 1 wherein said polar solvent is selected from the group consisting of: 2-methyl-1-propanol; methyl isoamyl ketone; n-butyl acetate; methyl isobutyl ketone; tetrahydrofuran; 2,6-lutidine; ethyl acetate; isopropanol; chloroform; cyclohexanone; methyl ethyl ketone; methyl n-propyl ketone; 2-picoline; dioxane; ethanol; nitroethane; pyridine; acetone; methoxyethanol; acetic acid; acetonitrile; methanol; nitromethane; m-cresol; and water.

14. The process of claim 1 wherein said polar solvent is methanol.

15. The process of claim 1 wherein said nonpolar solvent is a liquid solvent having a polarity index less than 4.

16. The process of claim 1 wherein said nonpolar solvent is selected from the group consisting of: squalane; isooctane; n-decane; 1,1,2-trichlorotrifluoroethane; cyclohexane; n-hexane; pentane; cyclopentane; heptane; petroleum ether; carbon disulfide; n-butyl chloride; carbon tetrachloride; dibutyl ether; triethylamine; diisopropyl ether; toluene; o-xylene; p-xylene; methyl t-butyl ether; bromobenzene; chlorobenzene; iodobenzene; o-dichlorobenzene; diethyl ether; benzene; dichloromethane; ethyl bromide; fluorobenzene; ethylene dichloride; isopentanol; ethylene chloride; 2-propanol; n-butanol; n-propanol; and tert.-butanol.

17. The process of claim 1 wherein said nonpolar solvent is dichloromethane.

18. The process of claim 1 wherein said nonpolar solvent is diethyl ether.

19. The process of claim 1 wherein said extraction conditions are maintained for an extraction period of from about one minute to three days.

20. The process of claim 19 wherein said extraction period is about one to 24 hours.

21. The process of claim 20 wherein said extraction period is about 24 hours.

22. The process of claim 21 wherein said extraction period is about 12 hours.

23. The process of claim 22 wherein said extraction period is about six hours.

24. The process of claim 1 wherein said extraction conditions are repeated one or more times.

25. The polar plant extract prepared by the process of claim 1.

26. The nonpolar plant extract prepared by the process of claim 1.

27. The extracted plant materials prepared by the process of claim 1.

28. A plastic-forming material derived from the polar plant extract of claim 25.

29. (canceled)

30. A preservative composition comprising the nonpolar extract of claim 26.

31. An antioxidant composition comprising the nonpolar extract of claim 26.

32. A fragrance ingredient comprising the nonpolar extract of claim 26.

33. A cleansing agent comprising the nonpolar extract of claim 26.

34. A disinfecting composition comprising the nonpolar extract of claim 26.

35-44. (canceled)

45. A preservative composition comprising the polar extract of claim 25.

46. An antioxidant composition comprising the polar extract of claim 25.

47. A cleansing agent comprising the polar extract of claim 25.

48. A disinfecting composition comprising the polar extract of claim 25.

49. The lignin extract of claim 6.

50-51. (canceled)

52. The tropolone extract of claim 6.

53. A preservative composition comprising the tropolone extract of claim 6.

54. A cleansing agent comprising the tropolone extract of claim 6.

55. A disinfecting composition comprising the tropolone extract of claim 6.

56. An antifungal composition comprising the tropolone extract of claim 6.

57. An insecticidal composition comprising the tropolone extract of claim 6.

58. An antibacterial composition comprising the tropolone extract of claim 6.

59-65. (canceled)
